(19) United States
(12) Patent Application Publication
(10) Pub. No.: US 2026/0275395 A1
(43) Pub. Date: Sep. 17, 2026

(54) ENZYMATIC CONVERSION OF D-XYLOSE INTO D-RIBOSE AND D-RIBULOSE-5-PHOSPHATE

(71) Applicants: Korea University Research and Business Foundation, Seoul (KR); Kookmin University Industry Academy Cooperation Foundation, Seoul (KR)

(72) Inventors: Kyoung Heon KIM, Seoul (KR); Yong Cheol PARK, Seoul (KR); Dong Hyun KIM, Seoul (KR); Ja Hyun LEE, Seoul (KR); Do Yeon KIM, Incheon (KR)

(73) Assignees: Korea University Research and Business Foundation, Seoul (KR); Kookmin University Industry Academy Cooperation Foundation, Seoul (KR)

(21) Appl. No.: 18/868,336

(22) PCT Filed: May 22, 2023
(86) PCT No.: PCT/KR2023/006915

§ 371 Date: Jan. 31, 2025

(30) Foreign Application Priority Data
May 25, 2022 (KR) 10-2022-0064225

(51) Int. Cl. C12P 19/24; C12N 9/12; C12N 9/90; C12P 19/02

(52) U.S. Cl. CPC C12P 19/24; C12N 9/1205; C12N 9/1229; C12N 9/90; C12P 19/02; C12Y 207/01016; C12Y 207/04001; C12Y 501/03; C12Y 503/01005; C12Y 503/01006

(57) ABSTRACT

The present invention relates to an enzymatic method for producing the energy sugars D-ribose and D-ribulose-5-phosphate at a high yield from D-xylose, which is a sugar commonly available in nature. By utilizing the present invention, it is possible to produce the high-value materials D-ribose and D-ribulose-5-phosphate economically and at high yields.

### TECHNICAL FIELD

The present invention relates to a method for efficiently producing D-ribose, an energy sugar, and D-ribulose-5-phosphate, a rare sugar derivative thereof, from D-xylose, a common sugar in nature, by constructing a bioprocess utilizing a multi-enzyme reaction and adenosine triphosphate (ATP) regeneration.

### BACKGROUND ART

Today, as income levels rise and consumers' awareness of health increases, the market for health functional foods or health supplements is growing. In addition, as the population enjoying leisure activities increases, various sports foods and drinks consumed during exercise are being released. In the United States, Europe, and Japan, sports foods have already become popular, making them easily available not only to athletes but also to the general public who need nutritional supplements. Among them, energy drinks are a representative example. Energy drinks are included in the category of functional drinks and are defined as drinks consumed for the purpose of replenishing energy. These energy drinks may be broadly classified into two types. First, energy drinks contain high-calorie sugars so that energy can be easily and quickly absorbed into the body. Second, energy drinks contain caffeine, taurine, and various amino acids to activate metabolism in the body. Most energy drinks on the market today belong to the latter, and when students or those who enjoy exercise consume excessive amounts, side effects such as caffeine dependence may occur.

D-ribose is a 5-carbon pentose sugar referred to as an energy sugar, and derivatives thereof are present in all living organisms. Examples thereof include adenosine triphosphate (ATP), which is a substance that provides energy, and related adenosine diphosphate (ADP), adenosine monophosphate (AMP), ribonucleic acid (RNA), deoxyribonucleic acid (DNA), and cofactors necessary for energy metabolism such as nicotinamide adenine dinucleotide (NAD)(H), nicotinamide adenine dinucleotide phosphate (NADP)(H), and flavin adenine dinucleotide (FAD). As such, D-ribose is a precursor and derivative of substances that play an important role in living organisms, and it exhibits various physiological activities when consumed. For example, D-ribose is an essential component of mitochondria in cells, and it is known to have the efficacy of improving cellular function as well as excellent recovery ability from exercise-induced stress and amelioration of cardiovascular diseases. It has also been reported to have an excellent skin improvement effect by improving ATP regeneration, so it can also be used as a functional cosmetic material.

D-ribulose-5-phosphate, a substance in which the fifth carbon of the keto-sugar D-ribulose is phosphorylated, is a major substrate of the pentose phosphate pathway that is widely present in animals, plants, and bacteria. Since the pentose phosphate pathway mainly provides reducing power and precursors essential for cell growth, D-ribulose-5-phosphate is a critical substance for investigating the principles and regulatory functions of the metabolic pathway. Therefore, D-ribulose-5-phosphate may be used as a precursor for the development of new drugs for inhibiting the pentose phosphate pathway of cancer cells and for the synthesis of 3-deoxy-D-manno-oct-2-ulosonic acid (KDO), which is a component of the lipoprotein of Gram-negative bacteria that is difficult to synthesize. However, D-ribulose-5-phosphate is currently sold at a high price of over about 100,000 KRW per mg, making it difficult to use in various studies.

Due to these possibilities, it is very promising industrially to implement a system capable of efficiently synthesizing D-ribose and D-ribulose-5-phosphate, one derivative thereof, using inexpensive starting materials.

Since it is difficult to synthesize D-ribose and D-ribulose-5-phosphate by a chemical method, attempts have been made to synthesize the two substances using bioengineering methods. D-ribose was produced using D-glucose, a hexose, as a substrate in genetically engineered microorganisms, but there were problems with low productivity and efficiency.

Therefore, the inventors of the present invention designed a multi-enzyme process that uses D-xylose, which is commonly found in nature, as a substrate to convert it into D-ribose and D-ribulose-5-phosphate, a derivative thereof and added an enzymatic process that regenerates ATP used in the process of synthesizing D-ribulose-5-phosphate to produce the two products at a high yield and in a cost-effective manner, thereby completing the present invention

### RELATED ART DOCUMENTS

### Non-Patent Documents

(Non-Patent Document 1) J Ind Microbiol Biotechnol (2015) 42:1117-1126

### DISCLOSURE

### Technical Problem

An object of the present invention is to provide an enzyme composition for producing D-ribose or D-ribulose-5-phosphate, a derivative thereof, using D-xylose as a substrate.

Another object of the present invention is to provide an enzymatic conversion method for producing D-ribose or D-ribulose-5-phosphate, a derivative thereof, using D-xylose as a substrate.

### Technical Solution

The present invention provides a novel enzymatic conversion method for producing D-ribose or D-ribulose-5-phosphate using D-xylose as a substrate.

The D-xylose is a pentose sugar like D-ribose and D-ribulose-5-phosphate, and is a major component of hemicellulose, one of the types of ligneous biomass that constitute the structure of a plant body. A plant body basically contains 25% to 35% (in the case of ligneous fiber) or 40% (in the case of herbaceous fiber) of D-xylose, so D-xylose is a very common sugar in nature.

Since the products targeted in the present invention are pentoses such as D-ribose and D-ribulose-5-phosphate, a derivative thereof, using another pentose having the same number of carbons as a substrate may obtain a higher theoretical yield than synthesizing it using a hexose such as glucose. In addition, it is a more suitable substrate in a situation where methods for minimizing carbon emissions during a process are in the spotlight to overcome climate change. Currently, the price of D-xylose is around 30,000 KRW per kg, and considering that the price of L-arabinose, another pentose substrate, is 2.2 million KRW per kg, D-xylose is a more suitable substrate from an economic perspective.

Accordingly, the present invention specifically provides a method of enzymatically producing D-ribose from D-xylose, the method including:
1) a step of converting D-xylose into D-xylulose by xylose isomerase using D-xylose as a substrate;
2) a step of converting D-xylulose into D-ribulose by ketose 3-epimerase using the produced D-xylulose as a substrate; and
3) a step of converting D-ribulose into D-ribose by ribose-5-phosphate isomerase using the produced D-ribulose as a substrate.

In addition, the present invention provides a method of enzymatically producing D-ribulose-5-phosphate from D-xylose, the method including:
1) a step of converting D-xylose into D-xylulose by xylose isomerase using D-xylose as a substrate;
2) a step of converting D-xylulose into D-ribulose by ketose 3-epimerase using the produced D-xylulose as a substrate; and
3) a step of converting ATP and D-ribulose into D-ribulose-5-phosphate by L-ribulokinase using the produced D-ribulose as a substrate.

In the method of enzymatically producing D-ribulose-5-phosphate, ATP may generally be used as a co-substrate for phosphate transfer in the process of converting D-ribulose into D-ribulose-5-phosphate, but ATP has a problem in that it is an expensive substrate and thus has low economic feasibility. Therefore, to overcome the above-described problem, the present inventors have found that when adenosine diphosphate (ADP) is enzymatically regenerated into ATP, the cost of ATP in the overall process can be significantly reduced, so that ADP and ATP are continuously circulated to regenerate ATP, which may be applied to the process of converting D-ribulose into D-ribulose-5-phosphate. Specifically, as enzymes used for enzymatic ATP regeneration, polyphosphate kinase, glycerol-3-phosphate kinase, acetic acid kinase, pyruvate kinase, carbamic acid kinase, and the like may be used without limitation, but preferably, polyphosphate kinase, capable of converting ADP into ATP using an inexpensive substrate hexametaphosphate (HMP), may be used.

In one specific embodiment of the present invention, it was confirmed that by using the polyphosphate kinase, ATP can be regenerated at a high yield while simultaneously producing the target product, D-ribulose-5-phosphate, at a high yield.

Each step of the enzymatic production method of synthesizing D-ribose or D-ribulose-5-phosphate from the D-xylose of the present invention may be performed sequentially by separating individual steps into multiple processes, or may be performed at once as a single process. In one specific embodiment, the present invention has made it possible to produce the target product at a high yield in a time- and cost-effective manner by selecting enzymes suitable for performing the synthesis of D-ribose or D-ribulose-5-phosphate from D-xylose in a single process and establishing the enzymatic conversion conditions.

The term “single process” used herein means that all intermediate processes for synthesizing D-ribose or D-ribulose-5-phosphate from D-xylose are performed in a single incubator. In other words, it may mean that various substrates and enzymes used for synthesizing the target product are injected into an incubator at once so that the process is performed.

Therefore, in the single process method of the present invention, since various enzymes are used, it is important to control the temperature conditions that may induce the activity of all enzymes, as well as the content and ratio of the substrates and enzymes.

The enzymatic single process of synthesizing D-ribose from D-xylose of the present invention is preferably performed at 40 to 55° C., and the enzymatic single process of synthesizing D-ribulose-5-phosphate from D-xylose is preferably performed at 30 to 45° C. When the temperature is outside the above-described range, the production yield of D-ribose or D-ribulose-5-phosphate may decrease due to a decrease in enzyme activity.

The concentration of D-xylose, which is the initial substrate for synthesizing D-ribose or D-ribulose-5-phosphate of the present invention, may be 1 to 10 mM, and preferably 5 to 10 mM.

In the method of enzymatically producing D-ribose from D-xylose of the present invention, the content ratio of the xylose isomerase:ketose 3-epimerase:ribose-5-phosphate isomerase may be 1:0.1 to 0.5:0.8 to 1 based on the unit of each enzyme.

In the method of enzymatically producing D-ribulose-5-phosphate from D-xylose of the present invention, in step 3), the content ratio of L-ribulokinase and polyphosphate kinase may be 1:0.5 to 2 based on the enzyme unit.

The xylose isomerase is an enzyme that converts the substrate D-xylose into D-xylulose, and may be xylose isomerase (BpXylA) derived from Bacteroides plebeius, but is not limited thereto. The xylose isomerase used in one specific embodiment may be represented by an amino acid sequence consisting of SEQ ID NO: 1 and encoded by a base sequence consisting of SEQ ID NO: 2.

The ketose 3-epimerase is an enzyme that converts the substrate D-xylulose into D-ribulose, and may be ketose 3-epimerase (AtDpe) derived from Agarobacterium tumefaciens, but is not limited thereto. The ketose 3-epimerase used in one specific embodiment may be represented by an amino acid sequence consisting of SEQ ID NO: 3 and encoded by a base sequence consisting of SEQ ID NO: 4.

The ribose-5-phosphate isomerase is an enzyme that converts the substrate D-ribulose into D-ribose, and may be D-ribose-5-phosphate isomerase (BpRpiB) derived from Bacteroides plebeius, but is not limited thereto. The D-ribose-5-phosphate isomerase used in one specific embodiment may be represented by an amino acid sequence consisting of SEQ ID NO: 5 and encoded by a base sequence consisting of SEQ ID NO: 6.

The L-ribulokinase is an enzyme that converts the substrate D-ribulose into D-ribulose-5-phosphate, and may be L-ribulokinase (EcAraB) derived from Escherichia coli, but is not limited thereto. The L-ribulokinase used in one specific embodiment may be represented by an amino acid sequence consisting of SEQ ID NO: 7 and encoded by a base sequence consisting of SEQ ID NO: 8.

The polyphosphate kinase is an enzyme that converts ADP into ATP using HMP as a substrate, and may be polyphosphate kinase (EcPpk) derived from Escherichia coli, but is not limited thereto. The polyphosphate kinase used in one specific embodiment may be represented by an amino acid sequence consisting of SEQ ID NO: 9 and encoded by a base sequence consisting of SEQ ID NO: 10.

The enzymes used in the synthesis of D-ribose or D-ribulose-5-phosphate from the D-xylose may be transcribed and translated through a DNA segment involved in producing a polypeptide, that is, a coding gene, including not only the regions before and after a coding region of an enzyme but also intervening sequences between individual coding segments. For example, the enzymes may be transcribed and translated from a nucleic acid sequence encoding the same, but are not particularly limited thereto. In addition, proteins having the activity of the enzymes as mutant proteins having one or more substitutions, deletions, translocations, additions, and the like of the enzymes are also included in the scope of right of the enzymes of the present invention, and preferably include an amino acid sequence having a sequence identity of 80% or more, 85% or more, 90% or more, 93% or more, 94% or more, 95% or more, 96% or more, 97% or more, 98% or more, and 99% or more with an amino acid sequence represented by any one of SEQ ID NOs: 1 to 5.

The enzymes may be obtained from Escherichia coli transformed with a recombinant vector containing a base sequence encoding the enzymes, or a culture thereof.

The terms “protein” and “polypeptide” are used interchangeably herein.

In the present invention, a polypeptide having a particular percentage (e.g., 80%, 85%, 90%, 95%, or 99%) of sequence identity with another sequence means that when the two sequences are aligned and compared, amino acid residues in that percentage are identical. The alignment and percent homology or identity may be determined using any suitable software program known in the art, such as those described in CURRENT PROTOCOLS IN MOLECULAR BIOLOGY (F. M. Ausubel et al. (eds) 1987 Supplement 30 section 7.7.18). Preferred programs include the GCG Pileup program, FASTA (Pearson et al. 1988 Proc. Natl Acad. Sci USA 85:2444-2448), and BLAST (BLAST Manual, Altschul et al., Natl. Cent. Biotechnol. Inf., Natl Lib. Med. (NCIB NLM NIH), Bethesda, MD, and Altschul et al. 1997 NAR 25:3389-3402). Another preferred alignment program is ALIGN Plus (Scientific and Educational Software, PA), preferably using default parameters. Still another available sequence software program is the TFASTA Data Searching Program available from the Sequence Software Package Version 6.0 (Genetics Computer Group, University of Wisconsin, Madison, WI).

The term “recombinant” when used in reference to a cell, nucleic acid, protein, or vector in the present invention means that the cell, nucleic acid, protein, or vector has been modified by the introduction of a heterologous nucleic acid or protein or by alteration of the native nucleic acid or protein, or that the cell is derived from a cell so modified. In other words, for example, a recombinant cell expresses a gene that is not found in the native (non-recombinant) form of the cell, or alternatively, expresses a native gene that is abnormally expressed when it is expressed, or does not express it at all.

The term “nucleic acid” as used herein encompasses single-stranded or double-stranded DNA, RNA, and chemical modifications thereof. The terms “nucleic acid” and “polynucleotide” may be used interchangeably herein. Since the genetic code is degenerated, one or more codons may be used to encode a particular amino acid, and the present invention encompasses polynucleotides encoding a particular amino acid sequence.

The term “introduction” of a nucleic acid sequence into a cell means “transfection” or “transformation,” or “transduction,” and includes reference to integration of a nucleic acid sequence into a eukaryotic or prokaryotic cell, and at this time, the nucleic acid sequence is integrated into the genome of the cell (e.g., a chromosome, plasmid, plastid, or mitochondrial DNA) and converted to an autonomous replicon, or is transiently expressed.

In addition, the present invention provides a composition for producing D-ribose, including:
an enzyme mixture including xylose isomerase, ketose 3-epimerase, and ribose-5-phosphate isomerase; and
D-xylose as a substrate.

In addition, the present invention provides a composition for producing D-ribulose-5-phosphate, including:
an enzyme mixture including xylose isomerase, ketose 3-epimerase, and L-ribulokinase; and
D-xylose as a substrate.

The composition for producing D-ribulose-5-phosphate may further include polyphosphate kinase for ATP regeneration, and the polyphosphate kinase may solve the cost problem that occurs when expensive ATP is directly used as a substrate, by converting ADP into ATP using HMP as a substrate.

Each enzyme of the composition for producing D-ribose or D-ribulose-5-phosphate may be used sequentially or simultaneously for the reaction, and specifically, may be used simultaneously for the production of the target product through a single process.

### Advantageous Effects

The present invention relates to an enzymatic method capable of producing D-ribose and D-ribulose-5-phosphate, which are energy sugars, at a high yield from D-xylose, a sugar commonly available in nature, and by using the present invention, D-ribose and D-ribulose-5-phosphate, which are high value-added materials, can be produced at a high yield and in a cost-effective manner.

### DESCRIPTION OF DRAWINGS

FIG. 1 shows a schematic diagram of a multi-enzyme reaction for converting D-xylose into D-ribose of the present invention.

FIG. 2 shows sodium dodecyl sulfate-polyacrylamide gel electrophoresis (SDS-PAGE) gel photographs confirming the expression and purification of the recombinant enzymes of the present invention. (a) of FIG. 2 shows SDS-PAGE gel photographs of BpXylA, AtDpe, and BpRpiB used in the multi-enzyme process for producing D-ribose from D-xylose, and (b) of FIG. 2 shows an SDS-PAGE gel photograph of BpXylA, AtDpe, EcAraB, and EcPpk producing D-ribulose-5-phosphate from D-xylose. The recombinant proteins are derived from Bacteroides plebeius, and the AtDpe recombinant protein is derived from Agarobacterium tumefaciens. Lastly, EcAraB and EcPpk are derived from Escherichia coli.

FIG. 3 shows the results about the basic characteristics of BpXylA of the present invention. (a) of FIG. 3 shows the optimum activity temperature, (b) of FIG. 3 shows the optimum activity pH, (c) of FIG. 3 shows the metal ion effect, and (d) of FIG. 3 shows the result of measuring the thermal stability.

FIG. 4 shows the results about the effect of metal ions on the thermal stability of BpXylA of the present invention. (a) of FIG. 4 shows the results of measuring the thermal stability by adding Mg²⁺, (b) of FIG. 4 shows the results of measuring the thermal stability by adding Co²⁺, and (c) of FIG. 4 shows the results of measuring the thermal stability by adding Mn²⁺.

FIG. 5 shows the results about the basic characteristics of BpXylA of the present invention. (a) of FIG. 5 shows the results of measuring the optimal activity temperature, (b) of FIG. 5 shows the results of measuring the optimal activity pH, (c) of FIG. 5 shows the results of measuring the metal ion effect, and (d) of FIG. 5 shows the results of measuring the thermal stability.

FIG. 6 shows the results of optimizing the usage amount of each of the recombinant enzymes to produce D-ribose from D-xylose. (a) of FIG. 6 shows the results of the production profile of the reaction product D-xylulose by varying the amount of BpXylA enzyme to investigate the optimal enzyme amount of BpXylA for the substrate xylose. (b) of FIG. 6 shows the results of the production profile of the reaction product D-ribulose from the substrate D-xylose by varying the amount of AtDpe enzyme to investigate the optimal amount of AtDpe enzyme together with the amount of BpXylA corresponding to the amount of BpXylA enzyme determined from the results of (a) of FIG. 6. (c) of FIG. 6 shows the results of the production profile of the final reaction product D-ribose from the substrate D-xylose by varying the amount of BpRpiB enzyme to investigate the optimal enzyme amount of BpRpiB together with amounts of BpXylA and AtDpe corresponding to the amounts of BpXylA and AtDpe enzymes determined from the results of (a) of FIG. 6 and (b) of FIG. 6.

FIG. 7 shows the production profile of the reaction product D-ribulose from the substrate D-xylose by varying the reaction temperature of the multi-enzyme process under the optimal enzyme amount conditions of BpXylA, AtDpe, and BpRpiB determined in [FIG. 6].

FIG. 8 shows a schematic diagram of the multi-enzyme reaction for converting D-xylose into D-ribulose-5-phosphate according to the present invention.

FIG. 9 shows the results about the basic characteristics of EcAraB of the present invention. (a) of FIG. 9 shows the results of measuring the optimal activity temperature, (b) of FIG. 9 shows the results of measuring the optimal activity pH, (c) of FIG. 9 shows the results of measuring the metal ion effect, and (d) of FIG. 9 shows the results of measuring the thermal stability.

FIG. 10 shows the results about the basic characteristics of EcPpk of the present invention. (a) of FIG. 10 shows the results of measuring the optimal activity temperature, (b) of FIG. 10 shows the results of measuring the optimal activity pH, (c) of FIG. 10 shows the results of measuring the metal ion effect, and (d) of FIG. 10 shows the results of measuring the thermal stability.

FIG. 11 shows the results about the relative production amount of D-ribulose-5-phosphate by varying the proportion of metal cofactors in the multi-enzyme process for producing D-ribulose-5-phosphate from D-xylose of the present invention.

FIG. 12 shows the results about the relative production amount of D-ribulose-5-phosphate by varying the total amount of two metal cofactors determined in the multi-enzyme process for producing D-ribulose-5-phosphate from D-xylose of the present invention.

FIG. 13 shows the results about the relative production amount of D-ribulose-5-phosphate by varying the concentration of HMP in the multi-enzyme process for producing D-ribulose-5-phosphate from D-xylose of the present invention.

FIG. 14 shows the results about the relative production amount of D-ribulose-5-phosphate by varying the concentration of ATP in the multi-enzyme process for producing D-ribulose-5-phosphate from D-xylose of the present invention.

FIG. 15 shows the results about the total turnover number (TTN) of ATP recycled through the regeneration process at each concentration when the concentration of ATP was varied in the multi-enzyme process for producing D-ribulose-5-phosphate from D-xylose of the present invention.

FIG. 16 shows the results about the relative production amount of D-ribulose-5-phosphate obtained by varying the ratio of EcPpk to EcAraB in the multi-enzyme process for producing D-ribulose-5-phosphate from D-xylose of the present invention.

FIG. 17 shows the results about the relative production amount of D-ribulose-5-phosphate obtained by varying the amounts of EcAraB and EcPpk to BpXylA and AtDpe in the multi-enzyme process for producing D-ribulose-5-phosphate from D-xylose of the present invention.

FIG. 18 shows the results about the production amount and yield of D-ribulose-5-phosphate obtained by varying the concentration of the substrate D-xylose in the multi-enzyme process for producing D-ribulose-5-phosphate from D-xylose of the present invention.

FIG. 19 shows the results about the relative production amount of D-ribulose-5-phosphate obtained by varying the total amount of enzymes in the multi-enzyme process for producing D-ribulose-5-phosphate from D-xylose of the present invention.

FIG. 20 shows the results about the yield of D-ribulose-5-phosphate obtained by varying the reaction temperature in the multi-enzyme process for producing D-ribulose-5-phosphate from D-xylose of the present invention.

### MODES OF THE INVENTION

Hereinafter, the present invention will be described in more detail through examples according to the present invention, but the scope of the present invention is not limited by the examples presented below.

### [Example 1] Cloning, Overexpression, and Purification of Recombinant Enzymes BpXyIA, AtDpe, BpRpiB, EcAraB and EcPpk Used in the Present Invention

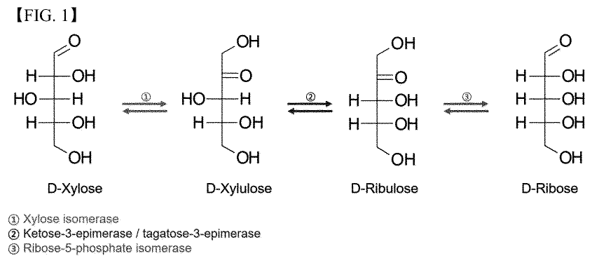

In the present invention, a multi-enzyme reaction was used to produce D-ribose, an energy sugar, using D-xylose, which is common in nature. As shown in [FIG. 1], xylose is converted into D-xylulose using xylose isomerase, D-xylulose is converted into D-ribulose using ketose 3-epimerase, and finally D-ribulose is converted into D-ribose using D-ribose-5-phosphate isomerase. For this series of reactions, xylose isomerase (BpXylA) and D-ribose-5-phosphate isomerase (BpRpiB) were cloned from Bacteroides plebeius, a gut microbe known to inhabit the human intestine, to obtain recombinant enzymes. In addition, an Agarobacterium tumefaciens-derived recombinant enzyme of ketose 3-epimerase (AtDpe), which has already been approved by the Ministry of Food and Drug Safety of Korea for D-psicose production, was secured.

In the multi-enzyme process for producing D-ribulose-5-phosphate from D-xylose, unlike the process for producing D-ribose, instead of using D-ribose-5-phosphate isomerase (BpRpiB) that converts D-ribulose to D-ribose, recombinant enzymes were obtained by cloning L-ribulokinase (EcAraB), which converts D-ribulose into D-ribulose-5-phosphate and ADP by consuming ATP, and polyphosphate kinase (EcPpk), which transfers phosphate molecules of polyphosphate salt to ADP to regenerate ATP. First, to obtain recombinant proteins of BpXylA and BpRpiB, genomic DNA of B. plebeius was extracted using a commercially available DNA isolation tool (Qiagen N.V., Hilden, Germany), and individual target genes BACPLE_02265 (accession number: B5CZU8) and BACPLE_02208 (accession number: B5CZP2) were amplified by polymerase chain reaction (PCR). At this time, a forward primer of ATACATATGATGGCAACAAAAGAGTACTTT (SEQ ID NO: 11) and a reverse primer of ATAGCGGCCGCGCAATACATATTTACAATAGCCTC (SEQ ID NO: 12) were used for BpXylA, and a forward primer of ATGCATATGATGAAAACAATA GGACTTGCATCCGACCA (SEQ ID NO: 13) and a reverse primer of ATAGCGGCCGCTCGAACGGGAATTTTGTCGATGCG (SEQ ID NO: 14) were used for BpRpiB. When cloning was performed to obtain the recombinant protein of AtDpe, a forward primer of ATACATATGAAACACGGCATCTATTATTCTTACTGGG (SEQ ID NO: 15) and a reverse primer of ATAGCGGCCGCGCCACCAAGAACGAAGCG (SEQ ID NO: 16) were used to amplify a target gene ATU4750 (accession number: A9CH28) by PCR.

To obtain recombinant proteins of EcAraB and EcPpk, genomic DNA of E. coli K12 was extracted using a commercially available DNA isolation tool (Qiagen N.V., Hilden, Germany), and individual target genes b0063 (accession number: P08204) and b2501 (accession number: POA7B1) were amplified by PCR. A forward primer of ATAGAGCTCATGGCGATTGCAATTGGCCT (SEQ ID NO: 17) and a reverse primer of ATAGCGGCCGCTAGAGTCGCAACGGCCTG (SEQ ID NO: 18) were used for EcAraB, and a forward primer of ATACATATGGGTCAGGAAAAGCTATACATCG (SEQ ID NO: 19) and a reverse primer of ATAGCGGCCGCTTCAGGTTGTTCGAGTGATTTG (SEQ ID NO: 20) were used for EcPpk. The PCR products obtained in this way were ligated into a pET21a vector, and the restriction enzymes used were NdeI and NotI except for EcAraB (SacI and NotI were used), and the recombinant plasmids produced in this way were transformed into Escherichia coli DH5a.

For overexpression of the obtained genes, recombinant plasmids were purified using the QIAprep Spin Miniprep tool (Qiagen), and then transformed into E. coli BL21 (DE3), which was a host for protein expression.

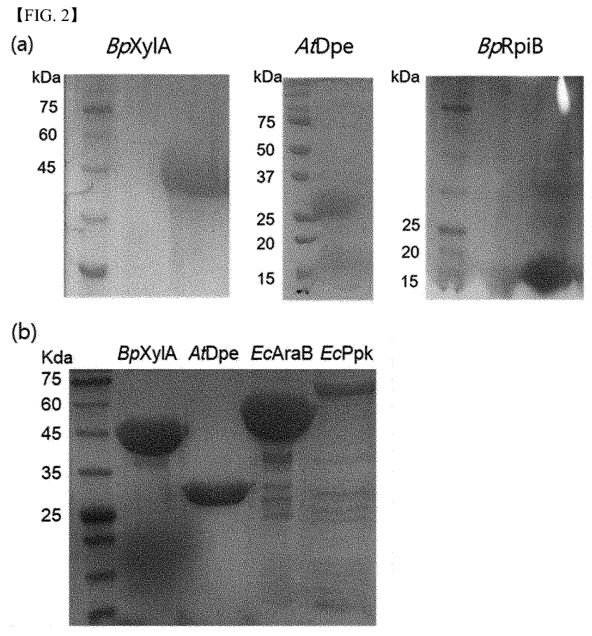

To overexpress the recombinant genes, E. coli BL21 (DE3) including each plasmid was cultured in a Luria-Bertani (LB) medium containing 100 μm/ml ampicillin at 37° C. until the absorbance at 600 nm reached 0.5, and then 0.5 mM isopropyl β-D-1-thiogalactopyranoside (IPTG) was added at an induction temperature of 16° C. to overexpress the proteins for 16 hours. Thereafter, cells were recovered through centrifugation, disrupted using an ultrasonicator (Branson Ultrasonics 450, Branson Ultrasonics Corp., Danbury, CT, USA), and centrifuged using a centrifuge (Combi-514R, Hanil Science Industrial CO. LTD., Daejeon, Korea) to obtain the supernatant. The supernatant was passed through a HisTrap column (GE Healthcare, Piscataway, USA) to purify the recombinant protein, and the size and degree of purification of the recombinant protein were confirmed using SDS-PAGE gel [(a) of FIG. 2 and (b) of FIG. 2], and the protein concentration was measured using a bicinchoninic acid (BCA) protein assay kit (Thermo Fisher Scientific, San Jose, CA, USA).

### [Example 2] Optimal Reaction Conditions for BpXylA

To investigate the optimal reaction conditions for BpXylA, the effects of optimal reaction temperature, pH, and metal ions were examined. The standard reaction conditions for BpXylA were set to a reaction with 1.09 U/ml BpXylA in 50 mM sodium phosphate (pH 7.0) buffer containing 10 mM D-xylose and 1 mM Mg²⁺ at 50° C. for 10 minutes. The optimal reaction conditions for BpXylA described below were all determined by experiments under the standard reaction conditions. The enzyme reaction was inactivated at 95° C. for five minutes, and the produced D-xylulose was quantified using an Aminex HPX-87H (300× 7.8 mm, Bio-Rad Inc., Hercules, California, USA) column to calculate the enzyme activity, and the amount (mg) of BpXylA that produces 1 μmol of D-xylulose per minute was defined as one unit.

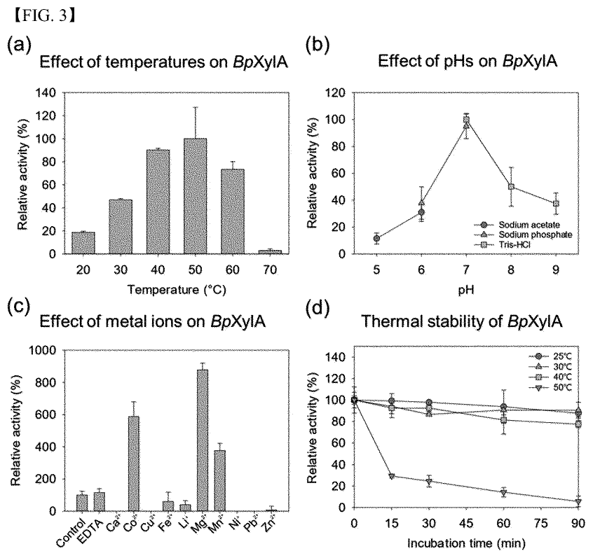

First, in order to determine the optimal temperature conditions for BpXylA, the reaction was performed under standard reaction conditions at various temperature conditions. As a result, as shown in [(a) of FIG. 3], the optimal temperature for enzyme activity of BpXylA was found to be 50° C. In order to determine the optimal pH of BpXylA, the reaction was performed while varying the pH using various buffer solutions. As shown in [(b) of FIG. 3], the highest activity was found in a sodium phosphate buffer at pH 7.0, which was determined as the optimal pH of BpXylA. In order to examine the effect of metal ions on the activity of BpXylA, a pre-incubation was performed at 4° C. for 20 minutes before the reaction, so that the enzyme and metal ions sufficiently reached an equilibrium, and the relative activity was measured using a condition where no metal ions were added was set as a control. As a result, as shown in [(c) of FIG. 3], the enzyme activity significantly increased under the conditions where Co²⁺, Mg²⁺, and Mn²⁺ were added, and in particular, the highest activity (876.98±35.30%) was found under the condition where Mg²⁺ was added. In order to examine the thermal stability of BpXylA, the enzyme was allowed to stand for a certain period of time under various temperature conditions, and then the reaction was performed under the standard reaction conditions. The decrease in enzyme activity due to heat treatment was determined by calculating the decrease in relative activity, using the activity of the enzyme that was not allowed to stand as a control. As a result, as shown in [(d) of FIG. 3], the enzyme activity did not significantly decrease for up to 90 minutes at 40° C., but it rapidly decreased to about 29.29±1.43% at 50° C. for 15 minutes. The optimal activity of BpXylA was found at 50° C., but the activity rapidly decreased at temperatures above 50° C. probably because the tertiary structure of the enzyme became unstable.

### [Example 3] Effect of Metal Ions on the Thermal Stability of BpXylA

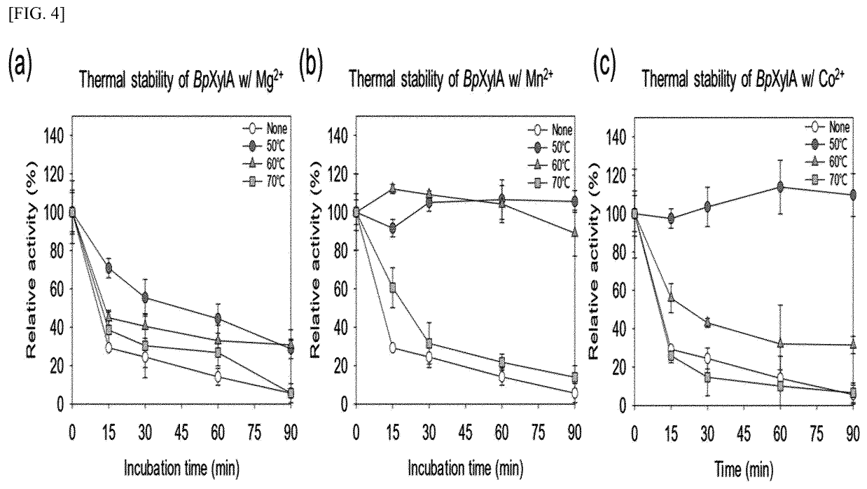

Since BpXylA exhibited high enzyme activity when Co²⁺, Mg²⁺, and Mn²⁺ were used as cofactors in [(d) of FIG. 3], the effect of the metal ions on the thermal stability of BpXylA was investigated. To investigate this, the culture was performed with each metal ion corresponding to a concentration of 1 mM together with the enzymes under various temperature conditions. As a result, as shown in [FIG. 4], higher thermal stability was found when the culture was performed with metal ions than when it was performed without metal ions. In the case of Mg²⁺ [(a) of FIG. 4], it was confirmed that the thermal stability increased, but the relative activity decreased over time at 50° C. or higher. In the cases of Co²⁺ and Mn²⁺, the relative activity was stably maintained for up to 90 minutes at 50° C., and in the case of Mn²⁺, the relative activity was found to be stable even at 60° C. [(b) of FIG. 4 and (c) of FIG. 4].

### [Example 4] Substrate Specificity of BpXyIA

In order to determine whether BpXylA used in the present invention is a suitable enzyme for producing D-ribose from D-xylose, the substrate specificity of BpXylA for various aldo- and keto-sugars was examined [Table 1]. The experiment was conducted using various aldo- and keto-sugars as substrates at a concentration of 2 mM under the standard reaction conditions, and the relative activity against other substrates was shown based on the activity for D-xylose. As a result, it was found that in the cases of aldo-sugars, BpXylA was active only against D-xylose and D-ribose, and in the case of keto-sugars, BpXylA was active only against D-xylulose and D-ribulose, which correspond to the aldo-sugars against which BpXylA is active. Therefore, BpXylA is sufficiently applicable for producing D-ribose from D-xylose in the present invention.

TABLE 1
Substrate specificity of BpXylA

| Substrate | | Relative activity (%) |
|---|---|---|
| Aldose | D-Arabinose | Not detected |
| | L-Arabinose | Not detected |
| | D-Glucose | Not detected |
| | D-Mannose | Not detected |
| | D-Ribose | 33.2 ± 1.1 |
| | D-Xylose | 100 ± 2.2 |
| Ketose | D-Fructose | Not detected |
| | D-Ribulose | 13.9 ± 13.8 |
| | L-Sorbose | Not detected |
| | D-Tagatose | Not detected |
| | D-Xylulose | 156.5 ± 2.2 |

### [Example 5] Optimal Reaction Conditions for BpRpiB

To determine the optimal reaction conditions for BpRpiB, the optimal reaction temperature, pH, metal ion effects, and thermal stability were examined. The standard reaction conditions for BpRpiB were set to a reaction with 0.8 U/ml of BpRpiB protein in a buffer solution containing 2 mM D-ribulose at 50° C. for 10 minutes, and the optimal reaction conditions for BpRpiB described below were all determined by experiments under the standard reaction conditions. The enzyme reaction was inactivated at 95° C. for five minutes, and the D-ribulose produced was measured for absorbance at 540 nm (xMark microplate reader, Bio-Rad Inc., Hercules, California, USA) by a cysteine-carbazole reaction to calculate the enzyme activity. The amount (mg) of BpRpiB that produces 1 μmol of D-ribulose per minute was defined as one unit.

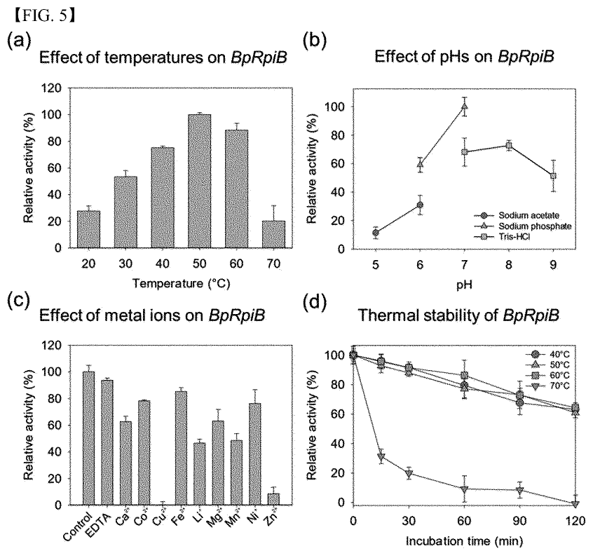

First, in order to determine the optimal temperature conditions for BpRpiB, the reaction was performed under standard reaction conditions at various temperature conditions. As a result, as shown in [(a) of FIG. 5], the highest activity was observed at 50° C. In order to investigate the optimal pH for the activity of BpRpiB, the reaction was performed by changing the pH using various buffer solutions. As a result, as shown in [(b) of FIG. 5], the highest activity was found in a sodium phosphate buffer (pH 7.0). In order to examine the effect of metal ions on the activity of BpRpiB, a pre-incubation was performed at 4° C. for 20 minutes before the reaction so that the enzyme and metal ions sufficiently reached equilibrium, and a condition where no metal ions were added was set as a control. As a result, as shown in [(c) of FIG. 5], there was no synergistic effect on the enzyme activity for a specific metal ion, and rather, the activity was significantly inhibited by Cu²⁺ and Zn²⁺, indicating that BpRpiB is an enzyme that does not use metal ions as a cofactor. In order to examine the thermal stability of BpRpiB, the enzyme was allowed to stand for a certain period of time under various temperature conditions, and then the reaction was performed under the standard reaction conditions. The decrease in enzyme activity due to heat treatment was determined by calculating the decrease in relative activity, using the activity of the enzyme that was not allowed to stand as a control. As a result, the overall activity of BpRpiB was stable up to 60° C., but the activity rapidly decreased at 70° C. [(d) of FIG. 5].

### [Example 6] Substrate Specificity of BpRpiB

BpRpiB used in the present invention is known to be an enzyme that converts the substrate D-ribulose-5-phosphate, which is originally a phosphorylated sugar, into D-ribose-5-phosphate. Therefore, in the present invention, in order to confirm the activity of BpRpiB for D-ribose and D-ribulose as well as D-ribulose-5-phosphate and D-ribose-5-phosphate, which are the original substrates of BpRpiB, the substrate specificity of BpRpiB for various aldo- and keto-sugars was examined [Table 2]. The experiment was performed using various aldo- and keto-sugars as substrates at a concentration of 2 mM under standard reaction conditions, and the relative activity against other substrates was expressed based on the activity against D-xylose. As a result, it was found that BpRpiB reacts only with D-ribose and D-ribulose, excluding the original substrates D-ribulose-5-phosphate and D-ribose-5-phosphate. Therefore, in the present invention, in order to produce D-ribose from D-xylose, BpRpiB is sufficiently applicable as an enzyme that converts D-ribulose into D-ribose, which is the final reaction step of the multi-enzyme process.

TABLE 2
Substrate specificity of BpRpiB

| Substrate | | Relative activity (%) |
|---|---|---|
| Aldose | D-Ribose-5-phosphate | 933.9 ± 11.7 |
| | D-Arabinose | Not detected |
| | L-Arabinose | Not detected |
| | D-Glucose | Not detected |
| | D-Mannose | Not detected |
| | D-Ribose | 79.1 ± 0.4 |
| | D-Xylose | Not detected |
| Ketose | D-Ribulose-5-phosphate | 924.4 ± 33.7 |
| | D-Fructose | Not detected |
| | D-Ribulose | 100 ± 4.8 |
| | L-Sorbose | Not detected |
| | D-Tagatose | Not detected |
| | D-Xylulose | Not detected |

### [Example 7] Selection of Optimal Usage Amount of Each Enzyme

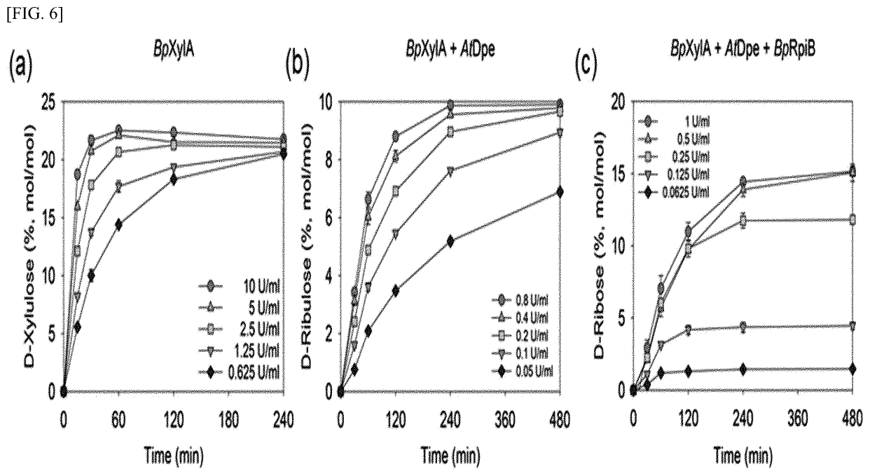

In a multi-enzyme process using multiple enzymes, since the reaction rates of each enzyme are different, specifying the appropriate enzyme amount and ratio of each enzyme amount according to the amount of substrate can reduce the process cost. In addition, since the multi-enzyme process designed to produce D-ribose in the present invention is performed using enzymes that all catalyze isomerization reactions, no more products are produced when each reactant and product reaches dynamic equilibrium. Therefore, in producing D-ribose from D-xylose, it was examined whether the reactants and products at each stage of the multi-enzyme reaction reached an equilibrium, and the optimal enzyme amount for each enzyme was determined so that the designed multi-enzyme process could reach an equilibrium state at which the yield is the theoretical maximum [FIG. 6].

The reaction conditions for the multiple enzymes included a 50 mM sodium phosphate (pH 7.0) buffer solution based on 10 g/L D-xylose. The reaction temperature was set to 45° C. to maintain the stability of the enzymes during the multi-enzyme process because the thermal stability of AtDpe decreased rapidly at 50° C. compared to 45° C., as found in a previous study. In the case of metal cofactors, the thermal stability of BpXylA greatly increased when Co²⁺ was used as a cofactor, and since AtDpe cannot use Mg²⁺ as a cofactor, Co²⁺ was selected as the cofactor for the enzymatic process. Each sugar (D-xylose, D-xylulose, D-ribulose, D-ribose) produced during the reaction was quantified using a Shodex SP0810 (8×300 mm, Showa Denko CO. LTD., Tokyo) column.

For the first reaction, which is conversion from D-xylose to D-xylulose, the reaction was performed at 45° C. with various concentrations of BpXylA with 10 g/L of D-xylose substrate, and as shown in [(a) of FIG. 6], it was confirmed that equilibrium was reached in 120 minutes at 2.5 U/ml. This is a shorter time than the 240 minutes set in the experiment, and therefore, even when only half of that amount, 1.25 U/ml of enzyme, was added, equilibrium would be sufficiently reached in 240 minutes, so this enzyme amount was selected. Although the reaction would reach an equilibrium even when only 0.625 U/ml of BpXylA was used, since the reaction time would be prolonged when this amount of enzyme was used, 1.25 U/ml of enzyme was selected for the first reaction.

The reaction of AtDpe for the second reaction, which is conversion from D-xylulose to D-ribulose, is one of a series of reactions that occur after the previous reaction of BpXylA. Therefore, in order to select the appropriate enzyme amount of AtDpe, the production profile of the reaction product D-ribulose was examined by performing the reaction with BpXylA at 1.25 U/ml, which was previously determined, and AtDpe at various concentrations using 10 g/L of D-xylose as a substrate. As a result, as shown in [(b) of FIG. 6], as the amount of added enzyme increased, the reaction rate for producing D-ribulose significantly increased. It was confirmed that until the condition of 0.1 U/ml, D-ribulose production did not reach an equilibrium even after 480 minutes, but when 0.4 U/ml of AtDpe was added, D-ribulose reached an equilibrium in 240 minutes. Therefore, 0.4 U/ml was selected as the appropriate amount of AtDpe to be used in this reaction.

Since the final reaction, which is conversion of D-ribulose to D-ribose, is also one of a series of reactions that occur after the previous reactions of the two enzymes, in order to select the appropriate enzyme amount of BpRpiB, the reaction was performed with various amounts of BpRpiB with BpXylA at 1.25 U/ml and AtDpe at 0.4 U/ml, which were previously determined, using 10 g/L of D-xylose as a substrate, and the production profile of the reaction product D-ribose was examined. As a result, as shown in [(c) of FIG. 6], it was confirmed that equilibrium was reached when 1 U/ml of BpRpiB was used, and the largest amount of D-ribose was produced. In this series of reactions, 1.69±0.03 g/L of D-ribose was produced from 10 g/L of D-xylose, which exhibited a yield of 16.88±0.32% based on the amount of added D-xylose.

### [Example 8] D-Ribose Production Yield and Equilibrium Relationship by Reaction Temperatures

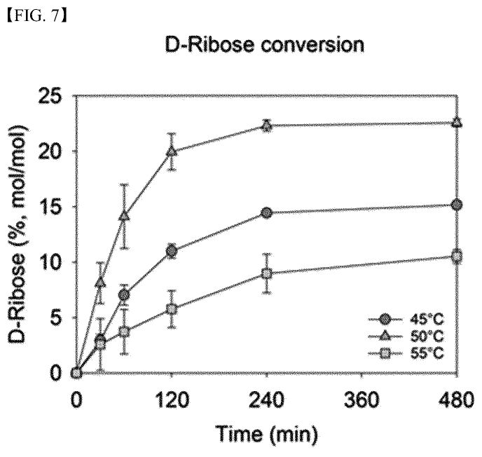

As shown in [Example 1], in the multi-enzyme reaction designed in the present invention, individual reactants and products form a chemical equilibrium relationship, and the equilibrium constant (Keq) representing this equilibrium relationship increases or decreases depending on the temperature. Therefore, it was expected that the D-ribose production yield of the multi-enzyme process would be dependent on the reaction temperature, so the production yield of D-ribose was investigated by conducting the reaction under the conditions of 45 to 55° C. [FIG. 7]. As a result, it was confirmed that when the temperature was increased from 45° C. to 50° C., the production yield of D-ribose increased from 16.88±0.32% to 25.26±0.32%, but the yield decreased to 10.17±0.49% at 55° C. or higher. As the reaction temperature increased, the equilibrium constant for producing D-ribose from the reactant D-xylose increased, thereby increasing the theoretical production yield of D-ribose. However, the activity of the AtDpe enzyme used in the multi-enzyme process was unstable above 50° C., and in particular, at a temperature above 55° C., the reaction was performed at a temperature outside the optimal activity range of the enzyme used in the process, so the yield was lower than when the reaction was performed at 50° C., which is 5° C. lower.

### [Example 9] Design of Multi-Enzyme Process Including D-Ribulose-5-Phosphate Production Using EcAraB and ATP Regeneration Using EcPpk

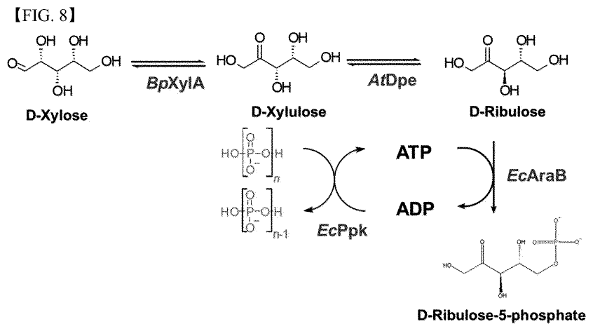

In order to produce D-ribulose-5-phosphate from D-xylose, the EcAraB enzyme was added instead of the BpRpiB enzyme in the multi-enzyme process performed in [Examples 1 to 8] [FIG. 8]. In this newly designed multi-enzyme process, D-xylose was converted into D-xylulose by BpXylA, D-xylulose was converted into D-ribulose by AtDpe, and D-ribulose was converted into ADP and D-ribulose-5-phosphate by EcAraB while consuming one ATP molecule. The produced ADP was regenerated to ATP by EcPpk using HMP as a substrate so that it could be used again as a substrate for EcAraB.

### [Example 10] Optimal Reaction Conditions for EcAraB

To determine the optimal reaction conditions for EcAraB, the optimal reaction temperature, pH, metal ion effects, and thermal stability were examined. The standard reaction conditions for EcAraB were set to a reaction with 0.0742 U/ml of EcAraB in a 50 mM Tris-HCl (pH 7.0) buffer solution containing 2 mM D-ribulose and 1 mM ATP at 50° C. for 10 minutes, and the optimal reaction conditions for EcAraB described below were all determined by experiments under the standard reaction conditions. The enzyme reaction was inactivated at 95° C. for five minutes, and then the produced D-ribulose-5-phosphate was quantified using an Aminex HPX-87H (300×7.8 mm, Bio-Rad Inc., Hercules, California, USA) column to calculate the enzyme activity. The amount (mg) of EcAraB that produces 1 μmol of D-ribulose-5-phosphate per minute was defined as one unit.

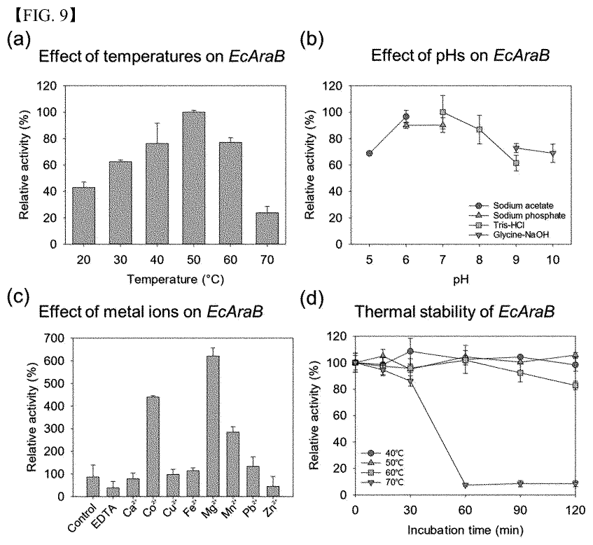

First, in order to determine the optimal temperature conditions for EcAraB, the reaction was performed under the standard reaction conditions at various temperature conditions. As a result, as shown in [(a) of FIG. 9], the highest activity was found at 50° C. In order to determine the optimal pH conditions for EcAraB, the reaction was performed while varying the pH using various buffer solutions. As a result, as shown in [(b) of FIG. 9], the highest activity was observed in Tris-HCl (pH 7.0), and the activity was close to 100% in a sodium phosphate buffer (pH 7.0). In order to examine the effect of metal ions on the activity of EcAraB, a pre-incubation was performed at 4° C. for 20 minutes before the reaction to ensure that the enzyme and metal ions sufficiently reached equilibrium, and a condition where no metal ions were added was set as a control. As a result, as shown in [(c) of FIG. 9], the maximum activity (614.62±42.14%) was observed with Mg²⁺, followed by high relative activities in the order of Co²⁺ (435.51±110.20%) and Mn²⁺ (286.47±21.62%). In order to examine the thermal stability of EcAraB, the enzyme was allowed to stand for a certain period of time at various temperature conditions, and then the reaction was performed under the standard reaction conditions. The decrease in enzyme activity was determined by calculating the decrease in relative activity due to heat treatment, using the activity of the enzyme that was not allowed to stand as a control. As a result, the overall activity of EcAraB was stable up to 60° C., but the activity was rapidly reduced at 70° C. [(d) of FIG. 9].

Regarding the substrate specificity of EcAraB, it was found in a previous study that EcAraB has activity against D-ribulose and D-xylulose. However, the activity against D-xylulose is only 2.44% relative to the activity against D-ribulose, so EcAraB is sufficiently applicable to produce D-ribulose-5-phosphate from D-ribulose in the present invention.

### [Example 11] Optimal Reaction Conditions for EcPpk

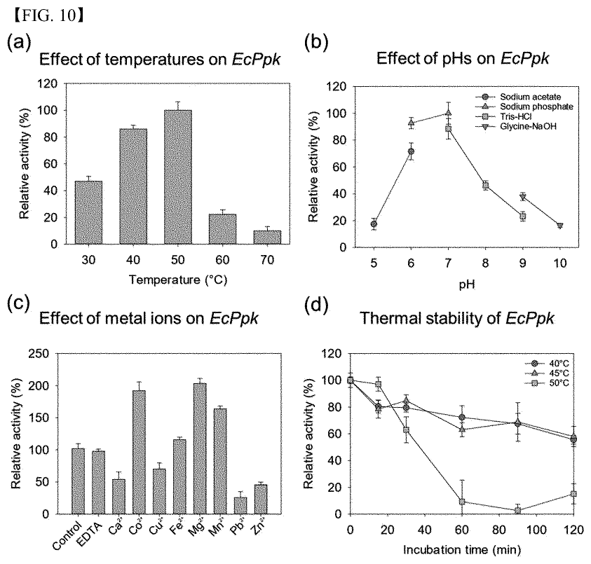

To determine the optimal reaction conditions for EcPpk, the optimal reaction temperature, pH, effect of metal ions, and thermal stability were examined. The standard reaction conditions for EcPpk were set to a reaction with 0.12 U/ml of EcPpk in a buffer solution containing 4 mM ADP and 5 mM HMP at 50° C. for 10 minutes, and the optimal reaction conditions for EcPpk described below were all determined through experiments under the standard reaction conditions. The enzyme reaction was inactivated at 95° C. for five minutes, and the produced ATP was quantified using an Aminex HPX-87H (300× 7.8 mm, Bio-Rad Inc., Hercules, California, USA) column to calculate the enzyme activity, and the amount (mg) of EcPpk that produces 1 μmol of ATP per minute was defined as one unit. First, to determine the optimal temperature conditions for EcPpk, the reaction was performed under the standard reaction conditions at various temperatures. As a result, the highest activity was found at 50° C., as shown in [(a) of FIG. 10]. In order to determine the optimal pH conditions for EcPpk, the reaction was performed by varying the pH using various buffer solutions. As a result, the highest activity was found in a sodium phosphate buffer (pH 7.0), as shown in [(b) of FIG. 10]. In order to investigate the effect of metal ions on the activity of EcPpk, pre-incubation was performed at 4° C. for 20 minutes before the reaction so that the enzyme and metal ions sufficiently reached equilibrium, and a condition where no metal ions were added was set as a control. As a result, as shown in [(c) of FIG. 10], the maximum activity (202.12±7.46%) was observed with Mg²⁺, followed by high relative activities in the order of Co²⁺ (176.14±24.10%) and Mn²⁺ (161.48±5.53%). In order to investigate the thermal stability of EcPpk, the enzyme was allowed to stand for a certain period of time under various temperature conditions, and then the reaction was performed under the standard reaction conditions. The decrease in enzyme activity was calculated by calculating the decrease in relative activity due to heat treatment, using the activity of the enzyme that was not allowed to stand as a control. As a result, the overall activity of EcPpk was stable up to 45° C., but the activity was rapidly reduced at 50° C. [(d) of FIG. 10].

### [Example 12] Setting the Optimal Reaction Conditions for Efficient D-Ribulose-5-Phosphate Production Utilizing ATP Regeneration

In [Example 12], an experiment was conducted to set the optimal conditions for each enzyme reaction for efficient production of D-ribulose-5-phosphate from D-xylose. In a multi-enzyme process using multiple enzymes, since each enzyme has different properties, specifying the appropriate amount of metal cofactors and substrates, the appropriate amount of each enzyme according to the amount of substrate added, and the ratio of each enzyme amount can reduce process costs. Therefore, in producing D-ribulose-5-phosphate from D-xylose, the amount of products generated under various conditions was confirmed, and the optimal amount of metal cofactors, substrates, and enzymes was determined so that the designed multi-enzyme process could achieve a high yield.

Unlike the D-ribose production by isomerization reaction, in this multi-enzyme process, when EcAraB phosphorylated D-ribulose using ATP, the equilibrium was significantly shifted from ATP to ADP, so the theoretically achievable production yield was expected to converge to almost 100%, achieving a much higher level of yield than the previous process. Therefore, in the present invention, the conditions that could affect the reaction when the individual enzymes were used together in the reaction were confirmed.

First, the reaction temperature of the multi-enzyme process was determined by referring to the optimal reaction temperature and thermal stability of the enzymes used in the multi-enzyme process. As shown in the results of [(d) of FIG. 9] and [(d) of FIG. 10], it was confirmed that the EcAraB enzyme exhibited optimal activity at 50° C. and was stable due to its high thermal stability, and the EcPpk enzyme exhibited optimal activity at 50° C., but most of the activity was lost in just one hour of reaction due to its low thermal stability at 50° C. However, it was confirmed that the activity was stably maintained up to 45° C., so the reaction temperature of the multi-enzyme process was selected to be 45° C., the same as the previously reported process. Regarding the optimal pH of the reaction, a 50 mM sodium phosphate (pH 7.0) buffer solution was selected because all enzymes exhibited the highest activity when the reaction was performed under the condition of a 50 mM sodium phosphate (pH 7.0) buffer solution.

The experiment to establish the optimal reaction conditions was set to include 5 mM D-xylose, 0.125 mM ATP, and 5 mM HMP substrate concentrations and 0.105 U/ml BpXylA, 0.03 U/ml AtDpe, 0.1 U/ml EcAraB, and 0.2 U/ml EcPpk in a 50 mM sodium phosphate (pH 7.0) buffer containing 10 mM Co²⁺ and 20 mM Mg²⁺ at 45° C. for 24 hours. The enzyme reaction was inactivated at 95° C. for five minutes, and then the amount of D-ribulose-5-phosphate produced was quantified using an Aminex HPX-87H (300× 7.8 mm, Bio-Rad Inc., Hercules, California, USA) column.

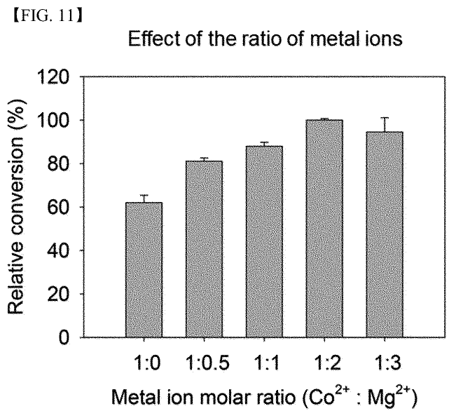

First, in order to determine the metal cofactor used in the reaction, the experiment was conducted by setting various Co²⁺ and Mg²⁺ ratios and total metal cofactor concentration according to the optimal ratio. Although EcAraB and EcPpk used in the multi-enzyme process exhibited the highest activity under the reaction condition using Mg²⁺ [(c) of FIG. 9] [(c) of FIG. 10], since AtDpe cannot use Mg²⁺ as a cofactor, Co²⁺ was also used to conduct the reactions. In order to examine the effect of the metal ion ratio (Co²⁺, Mg²⁺) on the multi-enzyme process, 5 to 30 mM Mg²⁺ was added to 10 mM Co²⁺ so that the Co²⁺:Mg²⁺ was 1:0.5 to 3, and the relative production amount of D-ribulose-5-phosphate was measured under each condition. The relative production amount was determined based on the largest amount of D-ribulose-5-phosphate as 100%. As a result, as shown in [FIG. 11], the production amount significantly increased under the condition where Mg²⁺ was also added compared to the condition where only Co²⁺ was added (62.02±3.40%), and in particular, the highest production amount (100.00±0.69%) was found under the condition where the concentration ratio of Co²⁺ and Mg²⁺ was 1:2 (10 mM Co²⁺, 20 mM Mg²⁺). However, it was confirmed that the production amount rather decreased (94.54±6.56%) under the condition where the concentration ratio of Co²⁺ and Mg²⁺ was 1:3 (10 mM Co²⁺, 30 mM Mg²⁺). Therefore, the condition where the concentration ratio of Co²⁺ and Mg²⁺ was 1:2 was set as the optimal condition.

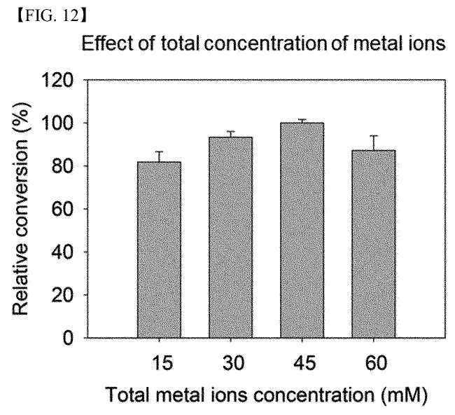

In this multi-enzyme process, metal cofactors are essentially used for the enzymatic reaction, and in order for each enzyme to react stably, there must be available metal cofactors at a certain concentration or higher. However, in this multi-enzyme process, since each phosphate group of HMP used as a substrate of EcPpk has a strong negative charge, a chelating phenomenon may occur, which may reduce the effect of the metal cofactor. In addition, when an excessive amount of metal cofactor is added to prevent this, the concentration of HMP used as a substrate of EcPpk may be reduced, and thus the production amount may be rather reduced. The results shown in [FIG. 11] where the production amount was reduced under the condition where the concentration ratio of Co²⁺ and Mg²⁺ was 1:3 were judged to be a representation of this phenomenon. Therefore, in the present invention, not only the ratio of metal cofactors but also the absolute concentrations of Co²⁺ and Mg²⁺ were compared in an experiment. In the experiment to determine the amount of metal cofactors for the multi-enzyme process, the relative production of D-ribulose-5-phosphate was measured by varying the sum of the concentrations of the two cofactors from 15 to 60 mM, while maintaining the concentration ratio of Co²⁺ and Mg²⁺ at 1:2. As a result, as shown in [FIG. 12], the highest value (100.00±1.63%) was found at the 45 mM condition, but it did not increase significantly compared to the 30 mM condition (93.32±2.72%), so the 30 mM condition was set to be the optimal condition.

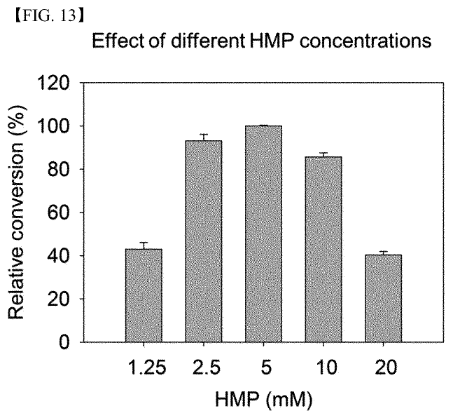

In order to determine the amount of HMP appropriate for the concentration of the determined metal cofactor, HMP was added at various concentrations (1.25 to 20 mM), and the relative production amount of D-ribulose-5-phosphate was measured under each condition. As a result, as shown in [FIG. 13], it was confirmed that the production amount was the highest under the 5 mM HMP condition (100.00±0.36%). The 10 mM condition showed a lower value (85.66±1.89%), so the 5 mM HMP condition was set as the optimal condition. Since HMP is a substrate of EcPpk, the ATP regeneration rate of EcPpk increases at high concentrations, which may increase the production amount. However, at concentrations above 5 mM, the inhibitory effect of HMP on other enzymes is greater, which is thought to result in a lower production amount.

In the multi-enzyme process of the present invention, as the ATP concentration increases, the ATP consumption rate of EcAraB increases, and the ADP generated through the reaction of EcAraB increases. When the amount of ADP increases, the ATP regeneration rate by EcPpk increases, so the amount of D-ribulose-5-phosphate produced may increase. From an economic perspective of the present invention, it is important to consume less ATP, which is an expensive substrate compared to D-xylose and HMP, and to regenerate it as much as possible. Considering the two above-described items, in order to examine the effect of ATP concentration on the multi-enzyme process, ATP was added at various concentrations (0.03125 to 0.5 mM) and the relative amount of D-ribulose-5-phosphate produced according to the ATP concentration was measured.

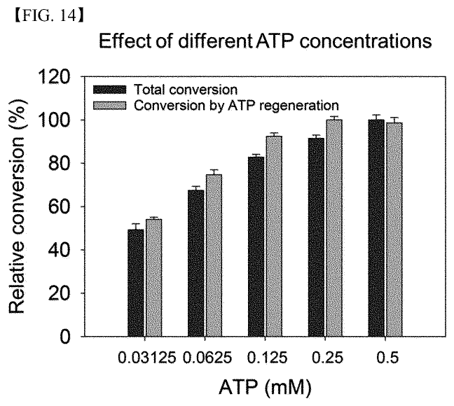

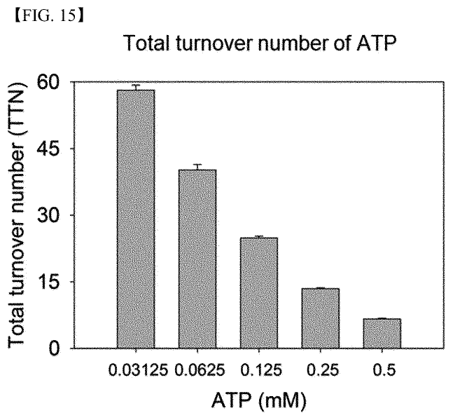

As a result, as shown in [FIG. 14], the total amount of D-ribulose-5-phosphate produced was the highest under the 0.5 mM ATP condition (100.00±2.33%), but the ATP regeneration index, i.e., the total turnover number (TTN) [FIG. 15], was the lowest (6.63±0.14%). As the concentration of ATP decreased, the TTN tended to continuously increase (6.63±0.14 to 58.14±0.93), but the total amount of D-ribulose-5-phosphate produced also decreased (100.00±2.33 to 49.31±2.78%). In contrast, the relative production amount of D-ribulose-5-phosphate by the regenerated ATP was the highest at 0.25 mM (100.00±1.23%) and increased from 0.03125 to 0.125 mM (54.06±0.86 to 91.42±1.30%), after which it did not significantly change even when the ATP concentration increased (0.25:100.00±1.23%, 0.5:98.60±2.06%). This is probably because the rate of ATP regeneration by EcPpk did not increase from 0.125 mM and because the ATP in the early stage of the reaction was consumed by EcAraB and only the ATP regenerated by EcPpk was used in the later stage. Although the production was highest under the 0.25 condition, there was no significant difference in production even when the concentration of ATP was increased from the 0.125 mM ATP condition. Therefore, considering that ATP is an expensive substrate, the 0.125 mM ATP condition was set to be the optimal condition.

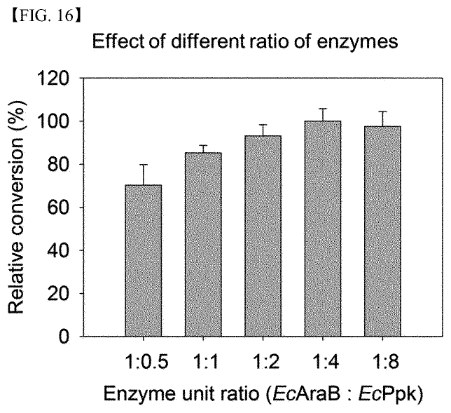

As mentioned above, when the ATP concentration is fixed in the multi-enzyme process, the total amount of D-ribulose-5-phosphate produced is affected by the ATP regeneration rate of EcPpk. Since the ATP regeneration rate varies depending on the ratio of EcPpk to EcAraB, the total amount of D-ribulose-5-phosphate produced may vary. In order to examine the effect of the ratio of the two enzymes on the multi-enzyme process, EcPpk (0.05 to 0.8 U/ml) was added to 0.1 U/ml EcAraB at the EcAraB:EcPpk ratio of 1:0.5 to 1:8, and the relative production amount of D-ribulose-5-phosphate was measured under each condition. As a result, as shown in [FIG. 16], it was confirmed that the production amount of D-ribulose-5-phosphate increased until the ratio of 1:0.5 to 1:2 (70.31±9.48 to 93.17±5.20%), but conditions where the ratio was higher than that (4:100.00±5.79%, 8:97.51±6.99%) showed no significant difference from the 1:2 condition. Since the amount of enzyme used in the multi-enzyme process is also an important criterion for the economic efficiency, the 1:2 condition was determined as the optimal condition based on the above-described results.

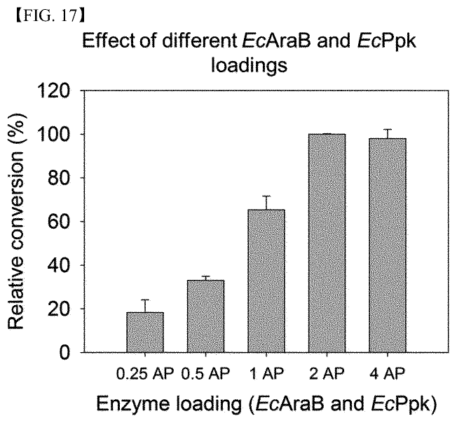

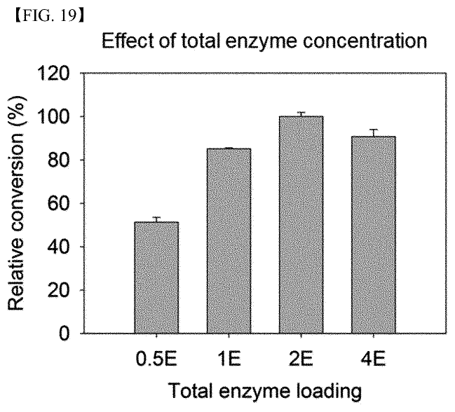

The phosphorylation of D-ribulose and the two enzymes that regenerate ATP may also affect the amount of D-ribulose-5-phosphate produced. When the amount of EcAraB increases, the rate of ATP consumption increases, and adding an appropriate ratio of EcPpk may also increase the rate of ATP regeneration, thereby increasing the amount of D-ribulose-5-phosphate produced. In order to examine the effect of the amounts of the two enzymes on the multi-enzyme process, while maintaining the ratio of EcAraB and EcPpk at 1:2, the absolute amounts of the two added enzymes were varied from 0.5 to 4 times the original amounts (0.05 to 0.4 U/ml EcAraB, 0.1 to 0.8 U/ml EcPpk), and the relative production amount of D-ribulose-5-phosphate was measured under each condition. As a result, as shown in [FIG. 17], the production amount of D-ribulose-5-phosphate showed the highest production rate (100.00±0.23%) under the condition where the amounts of the two enzymes were doubled, and the production rate remained the same thereafter (AP4: 98.02±4.18%). Therefore, the above condition was set to be the optimal condition, and 0.2 U/ml EcAraB and 0.4 U/ml EcPpk were used in the experiments conducted after this experiment. In addition, since the total amount of four enzymes used in the multi-enzyme process may also increase the reaction rate, thereby increasing the amount of D-ribulose-5-phosphate produced during the entire reaction time, an experiment was conducted by varying the total amount of enzymes. To examine the effect of the total enzyme amount on the multi-enzyme process, the above-mentioned condition was set to 1, and enzymes corresponding to 0.5, 1, 2, and 4-fold amounts were added (0.0525-0.42 U/ml BpXylA, 0.015 to 0.12 U/ml AtDpe, 0.1 to 0.8 U/ml EcAraB, 0.2-1.6 U/ml EcPpk), and the relative production amount of D-ribulose-5-phosphate under each condition was measured. As a result, as shown in [FIG. 19], it was confirmed that the value increased (53.89±2.23 to 100.00±1.94%) under the 0.5- to 2-fold enzyme amount conditions, and the largest amount of D-ribulose-5-phosphate was produced in the 2-fold condition. Under the condition in which the enzyme amount is increased 4-fold, the relative production of D-ribulose-5-phosphate rather decreased to 90.71±3.32%, and the 2-fold enzyme amount condition was determined as the optimal condition.

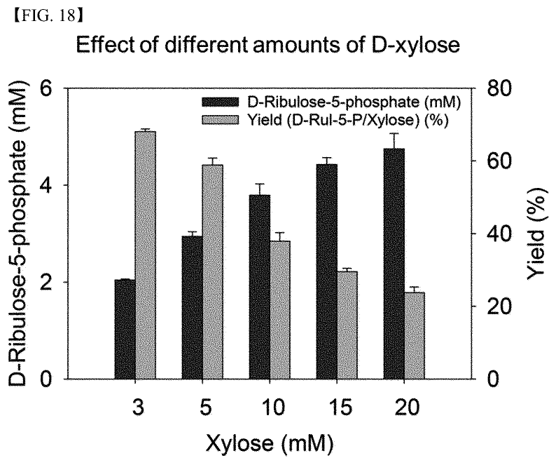

Using the optimized amounts of each enzyme under the above-described conditions, the production amount (in mM) of D-ribulose-5-phosphate was confirmed by varying the concentration of the substrate D-xylose. In order to examine the effect of D-xylose concentration on the multi-enzyme process, the concentration of D-xylose was increased to 3 to 20 mM and the production amount of D-ribulose-5-phosphate was measured under each condition. As a result, as shown in [FIG. 18], as the concentration of D-xylose increased, the production amount of D-ribulose-5-phosphate also increased (2.04±0.02 to 4.75±0.32 mM) under the condition, but the yield for the D-xylose substrate input into the reaction decreased (68.04±0.61 to 23.74±1.30%), confirming that D-xylose substrate concentrations higher than 5 mM did not significantly contribute to the increase in the production amount of D-ribulose-5-phosphate.

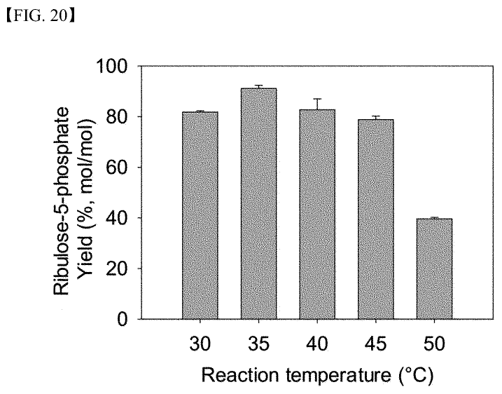

In a multi-enzyme reaction, since enzymes become unstable and their activity decreases over time, not only the optimal reaction rate of the enzymes but also their stability may have a significant impact on the yield of the entire process. In addition, since the amount of energy required for the reaction decreases as the reaction is performed at a lower temperature, there is an advantage in terms of cost and energy. In the multi-enzyme process designed in the present invention, isomerases are used up to the reaction of converting D-xylose into D-ribulose via D-xylulose, so there is a thermodynamic equilibrium relationship between the products. However, in the final stage of the multi-enzyme process, in which D-ribulose is converted into D-ribulose-5-phosphate, the energy difference of the reaction is large, so the thermodynamic equilibrium may not be considered. Therefore, unlike the multi-enzyme process for D-ribose production in which the yield increases as the temperature increase, more advantageous reaction conditions are formed as long as sufficient enzyme activity and stability are maintained even at a low temperature. Therefore, in order to examine the effect of temperature conditions on the multi-enzyme process optimized in [Example 12] of the present invention, a reaction was performed under various temperature conditions (30 to 50° C.), and the relative production amount of D-ribulose-5-phosphate was measured under each condition. As a result, it was confirmed that the highest production amount (91.14±1.24%) was achieved at 35° C., and the TTN at this time was 35.62±0.4. A drastic decrease in the yield (39.57±0.61%) was confirmed at 50° C. (FIG. 20).

1. A composition for producing D-ribose, comprising:
an enzyme mixture including xylose isomerase, ketose 3-epimerase, and ribose-5-phosphate isomerase; and
D-xylose as a substrate.

2. A composition for producing D-ribulose-5-phosphate, comprising:
an enzyme mixture including xylose isomerase, ketose 3-epimerase, and L-ribulokinase; and
D-xylose as a substrate.

3. The composition of claim 2, wherein the enzyme mixture further includes polyphosphate kinase and further includes adenosine diphosphate (ADP) and hexametaphosphate (HMP), which is a substrate of polyphosphate kinase.

4. The composition of claim 1, wherein each enzyme of the enzyme mixture is used sequentially or simultaneously.

5. A method of enzymatically producing D-ribose from D-xylose, the method comprising:
1) a step of converting D-xylose into D-xylulose by xylose isomerase using D-xylose as a substrate;
2) a step of converting D-xylulose into D-ribulose by ketose 3-epimerase using the produced D-xylulose as a substrate; and
3) a step of converting D-ribulose into D-ribose by ribose-5-phosphate isomerase using the produced D-ribulose as a substrate.

6. A method of enzymatically producing D-ribulose-5-phosphate from D-xylose, the method comprising:
1) a step of converting D-xylose into D-xylulose by xylose isomerase using D-xylose as a substrate;
2) a step of converting D-xylulose into D-ribulose by ketose 3-epimerase using the produced D-xylulose as a substrate; and
3) a step of converting ATP and D-ribulose into D-ribulose-5-phosphate by L-ribulokinase using the produced D-ribulose as a substrate.

7. The method of claim 6, wherein in step 3), ATP is produced from ADP by polyphosphate kinase using HMP as a substrate.

8. The method of claim 5, wherein a content ratio of the xylose isomerase:ketose 3-epimerase:ribose-5-phosphate isomerase is 1:0.1 to 0.5:0.8 to 1 based on the unit of each enzyme.

9. The method of claim 7, wherein in step 3), a content ratio of L-ribulokinase and polyphosphate kinase is 1:0.5 to 2 based on the enzyme unit.

10. The method of claim 6, wherein in step 1), the concentration of the substrate D-xylose is 1 to 10 mM.

11. The method of claim 5, wherein the method is performed at 40 to 55° C.

12. The method of claim 6, wherein the method is performed at 30 to 45° C.

13. The method of claim 5, wherein each step of the method is performed as a single process.

14. The method of claim 5, wherein the xylose isomerase consists of an amino acid sequence represented by SEQ ID NO: 1, and the ketose 3-epimerase consists of an amino acid sequence represented by SEQ ID NO: 3.

15. The method of claim 5, wherein the ribose-5-phosphate isomerase consists of an amino acid sequence represented by SEQ ID NO: 5.

16. The method of claim 6, wherein the L-ribulokinase consists of an amino acid sequence represented by SEQ ID NO: 7.

17. The method of claim 7, wherein the polyphosphate kinase consists of an amino acid sequence represented by SEQ ID NO: 9.


SEQUENCE LISTING (20 sequences)

SEQ ID NO: 1 - protein, 438 aa, synthetic construct
  REGION 1..438: note = BpXylA
MATKEYFPGIGKIKFEGVESKNPMAFRYYNAEKVILGKPMKEWLKFAMAWWHTLCAEGGDQFGGGTKSFPWKGNPDKVQAAKDKVDAGFEFMQKIGIEYYCFHDVDLCDEAATIEEYEANLKEVVAYLKQKQAETGIKLLWGTANVFGHARYMNGAATNPDFDVVARAAVQIKNAIDATIELGGTNYVFWGGREGYMSLLNTDQKREKEHLAQMLKLARDYARSKGFQGTFLIEPKPMEPSKHQYDVDTETVIGFLKAHGLDKDFKVNIEVNHATLAGHTFEHELAVAVDNNMLGSIDANRGDYQNGWDTDQFPIDNYELIQAMMQIIRGGGLGNGGTNFDAKTRRNSTDLEDIFIAHIAGMDAMARALENAARLLEESPYKKMLADRYASFDNGKGKEFEEGKLTLEDVVAYAKTVGEPKQTSGKQELYEAIVNMYC

SEQ ID NO: 2 - DNA, 1317 bp, synthetic construct
  misc_feature 1..1317: note = BpXylA
  source 1..1317: mol_type = other DNA
atggcaacaaaagagtactttccgggtattggaaaaatcaaatttgaaggtgtagaaagcaaaaacccgatggctttccgttattataatgcagaaaaagtcatcctgggcaaaccgatgaaagaatggttgaaattcgctatggcatggtggcacacattgtgcgctgaaggtggcgaccagttcggtggcggaacaaagagcttcccatggaaaggaaatcctgacaaagtacaggctgcaaaagataaagtagatgcaggttttgaattcatgcagaaaatcggtatcgaatactactgcttccacgatgtagacttgtgcgacgaagctgctactatcgaagaatacgaagctaacttgaaagaagtagtagcttacctgaaacagaaacaggctgaaactggtatcaaactgttgtggggtactgccaacgtattcggtcatgcacgttatatgaacggtgcagctactaaccctgactttgatgtagtagctcgtgctgccgtacagattaagaacgccatcgatgcaactatcgaactgggcggtacaaactacgtattctggggtggccgtgaaggttacatgtcactgctgaacactgaccagaaacgtgaaaaagaacacttggctcagatgttgaaactggctcgtgactatgcacgcagcaaaggtttccagggtactttcctgattgaaccgaaaccgatggaaccaagcaagcaccagtacgacgtagatactgaaactgttatcggcttcctgaaagcacacggcctggacaaagacttcaaggtaaatatcgaagtaaaccacgctactttggctggtcacacatttgaacacgaactggcagtagctgtagacaacaacatgctgggttctatcgatgctaaccgtggtgactaccagaacggatgggatactgaccagttccctatcgacaactatgaactgattcaggctatgatgcagattatccgtggtggtggtctgggcaacggtggtacaaacttcgatgcgaagactcgtcgtaactctactgacctggaagacatcttcatcgctcacatcgctggtatggatgccatggcacgtgctttggaaaatgcagcacgcctgctggaagaatctccttacaagaaaatgctggctgaccgctacgcttcattcgacaacggaaaaggtaaagaatttgaagaaggcaagctgacactggaagatgtagtagcttatgcaaagactgtaggtgaaccgaaacagaccagtggaaaacaagaactgtatgaggctattgtaaatatgtattgctaa

SEQ ID NO: 3 - protein, 289 aa, synthetic construct
  REGION 1..289: note = AtDpe
MKHGIYYSYWEHEWSAKFGPYIEKVAKLGFDIIEVAAHHINEYSDAELATIRKSAKDNGIILTAGIGPSKTKNLSSEDAAVRAAGKAFFERTLSNVAKLDIHTIGGALHSYWPIDYSQPVDKAGDYARGVEGINGIADFANDLGINLCIEVLNRFENHVLNTAAEGVAFVKDVGKNNVKVMLDTFHMNIEEDSFGDAIRTAGPLLGHFHTGESNRRVPGKGRMPWHEIGLALRDINYTGAVIMEPFVKTGGTIGSDIKVWRDLSGGADIAKMDEDARNALAFSRFVLGG

SEQ ID NO: 4 - DNA, 870 bp, synthetic construct
  misc_feature 1..870: note = AtDpe
  source 1..870: mol_type = other DNA
atgaaacacggcatctattattcttactgggaacatgagtggagcgccaagttcggtccctatatcgagaaggtcgccaagctcggtttcgacatcatcgaagtcgccgcccaccatatcaacgaatacagcgacgccgaactcgcgaccatcaggaagagcgcgaaggataacggcatcatcctcaccgccggcatcggtccgtcgaaaaccaagaacctgtcgtcggaagatgctgcggtgcgtgcggccggcaaggcgttctttgaaagaaccctttcgaacgtcgccaagctcgatatccacaccatcggcggcgcattgcattcctattggccaatcgattattcgcagcccgtcgacaaggcaggcgattatgcgcgcggcgtcgagggtatcaacggcattgccgatttcgccaatgatctcggcatcaacctgtgcatcgaagtcctcaaccgctttgaaaaccacgtcctcaacacggcggcggaaggcgtcgcttttgtgaaggatgtcggcaagaacaatgtgaaagtcatgctggataccttccacatgaacatcgaggaagacagtttcggtgacgccatccgcacggccggcccgcttctggggcacttccataccggtgaaagcaatcgccgcgtaccgggcaagggcagaatgccgtggcacgaaatcggccttgcgctgcgtgatatcaactacaccggcgcggtaatcatggagcctttcgtcaagacaggcggcaccatcggctcggatatcaaggtgtggcgcgacctgagcggtggcgccgacatcgcgaaaatggatgaagatgcccgcaatgcgctggcattctcccgcttcgttcttggtggctga

SEQ ID NO: 5 - protein, 144 aa, synthetic construct
  REGION 1..144: note = BpRpiB
MKTIGLASDHAGFELKQYVKKWLEAKGWEYKDFGTYTTDSCDYPDFAHPLALAVENGECYPGIAICGSGEGIGITLNKHQGIRAALCWIPEIAHLARQHNNANVLVMPGRFIDEAMADKIMTEFFTTDFEGGRHQARIDKIPVR

SEQ ID NO: 6 - DNA, 435 bp, synthetic construct
  misc_feature 1..435: note = BpRpiB
  source 1..435: mol_type = other DNA
atgaaaacaataggacttgcatccgaccatgccggatttgaattgaaacaatacgtgaaaaaatggctggaagccaaaggatgggaatacaaagatttcggaacttacacaaccgacagttgtgactatcccgactttgctcacccgctggctttggcagtagaaaacggagaatgctatccgggaattgccatctgcggaagtggtgaaggaatcggtatcaccctgaacaaacaccaaggtatccgtgccgcactttgctggattccggaaatcgctcatctggcccgtcagcacaacaatgccaacgtactggtgatgccgggacgtttcattgatgaagcaatggccgacaagatcatgacggaattcttcactaccgattttgaaggcggacgccatcaggcacgcatcgacaaaattcccgttcgataa

SEQ ID NO: 7 - protein, 566 aa, synthetic construct
  REGION 1..566: note = EcAraB
MAIAIGLDFGSDSVRALAVDCASGEEIATSVEWYPRWQKGQFCDAPNNQFRHHPRDYIESMEAALKTVLAELSVEQRAAVVGIGVDSTGSTPAPIDADGNVLALRPEFAENPNAMFVLWKDHTAVERSEEITRLCHAPGNVDYSRYIGGIYSSEWFWAKILHVTRQDSAVAQSAASWIELCDWVPALLSGTTRPQDIRRGRCSAGHKSLWHESWGGLPPASFFDELDPILNRHLPSPLFTDTWTADIPVGTLCPEWAQRLGLPESVVISGGAFDCHMGAVGAGAQPNALVKVIGTSTCDILIADKQSVGERAVKGICGQVDGSVVPGFIGLEAGQSAFGDIYAWFGRVLSWPLEQLAAQHPELKAQINASQKQLLPALTEAWAKNPSLDHLPVVLDWFNGRRSPNANQRLKGVITDLNLATDAPLLFGGLIAATAFGARAIMECFTDQGIAVNNVMALGGIARKNQVIMQACCDVLNRPLQIVASDQCCALGAAIFAAVAAKVHADIPSAQQKMASAVEKTLQPRSEQAQRFEQLYRRYQQWAMSAEQHYLPTSAPAQAAQAVATL

SEQ ID NO: 8 - DNA, 1701 bp, synthetic construct
  misc_feature 1..1701: note = EcAraB
  source 1..1701: mol_type = other DNA
atggcgattgcaattggcctcgattttggcagtgattctgtgcgagctttggcggtggactgcgccagcggtgaagagatcgccaccagcgtagagtggtatccccgttggcaaaaagggcaattttgtgatgccccgaataaccagttccgtcatcatccgcgtgactacattgagtcaatggaagcggcactgaaaaccgtgcttgcagagcttagcgtcgaacagcgcgcagctgtggtcgggattggcgttgacagtaccggctcgacgcccgcaccgattgatgccgacggtaacgtgctggcgctgcgcccggagtttgccgaaaacccgaacgcgatgttcgtattgtggaaagaccacactgcggttgaaagaagcgaagagattacccgtttgtgccacgcgccgggcaatgttgactactcccgctatattggcggtatttattccagcgaatggttctgggcaaaaatcctgcatgtgactcgccaggacagcgccgtggcgcaatctgccgcatcgtggattgagctgtgcgactgggtgccagctctgctttccggtaccacccgcccgcaggatattcgtcgcggacgttgcagcgccgggcataaatctctgtggcacgaaagctggggcggcttgccgccagccagtttctttgatgagctggacccgatcctcaatcgccatttgccttccccgctgttcactgacacctggactgccgatattccggtgggcaccttatgcccggaatgggcgcagcgtctcggcctgcctgaaagcgtggtgatttccggcggcgcgtttgactgccatatgggcgcagttggcgcaggcgcacagcctaacgcactggtaaaagttatcggtacttccacctgcgacattctgattgccgacaaacagagcgttggcgagcgggcagttaaaggtatttgcggtcaggttgatggcagcgtggtgcctggatttatcggtctggaagcaggccaatcggcgtttggtgatatctacgcctggttcggtcgcgtactcagctggccgctggaacagcttgccgcccagcatccggaactgaaagcgcaaatcaacgccagccagaaacaactgcttccggcgctgaccgaagcatgggccaaaaatccgtctctggatcacctgccggtggtgctcgactggtttaacggtcgtcgctcgccaaacgctaaccaacgcctgaaaggggtgattaccgatcttaacctcgctaccgacgctccgctgctgttcggcggtttgattgctgccaccgcctttggcgcacgcgcaatcatggagtgctttaccgatcaggggatcgccgtcaataacgtgatggcgctgggcggcatcgcgcggaaaaaccaagtcattatgcaggcctgctgcgacgtgctgaatcgcccgctgcaaattgttgcctctgaccagtgctgtgcgctcggtgcggcgatttttgctgccgtcgccgcgaaagtgcacgcagacatcccatcagcccagcaaaaaatggccagtgcggtagagaaaaccctgcaaccgcgcagcgaacaggcacaacgctttgaacagctttatcgccgctatcagcaatgggcgatgagcgccgaacaacactatcttccaacttccgccccggcacaggctgcccaggccgttgcgactctataa

SEQ ID NO: 9 - protein, 687 aa, synthetic construct
  REGION 1..687: note = EcPpk
MGQEKLYIEKELSWLSFNERVLQEAADKSNPLIERMRFLGIYSNNLDEFYKVRFAELKRRIIISEEQGSNSHSRHLLGKIQSRVLKADQEFDGLYNELLLEMARNQIFLINERQLSVNQQNWLRHYFKQYLRQHITPILINPDTDLVQFLKDDYTYLAVEIIRGDTIRYALLEIPSDKVPRFVNLPPEAPRRRKPMILLDNILRYCLDDIFKGFFDYDALNAYSMKMTRDAEYDLVHEMEASLMELMSSSLKQRLTAEPVRFVYQRDMPNALVEVLREKLTISRYDSIVPGGRYHNFKDFINFPNVGKANLVNKPLPRLRHIWFDKAQFRNGFDAIRERDVLLYYPYHTFEHVLELLRQASFDPSVLAIKINIYRVAKDSRIIDSMIHAAHNGKKVTVVVELQARFDEEANIHWAKRLTEAGVHVIFSAPGLKIHAKLFLISRKENGEVVRYAHIGTGNFNEKTARLYTDYSLLTADARITNEVRRVFNFIENPYRPVTFDYLMVSPQNSRRLLYEMVDREIANAQQGLPSGITLKLNLVDKGLVDRLYAASSSGVPVNLLVRGMCSLIPNLEGISDNIRAISIVDRYLEHDRVYIFENGGDKKVYLSSADWMTRNIDYRIEVATPLLDPRLKQRVLDIIDILFSDTVKARYIDKELSNRYVPRGNRRKVRAQLAIYDYIKSLEQPE

SEQ ID NO: 10 - DNA, 2067 bp, synthetic construct
  misc_feature 1..2067: note = EcPpk
  source 1..2067: mol_type = other DNA
atgggtcaggaaaagctatacatcgaaaaagagctcagttggttatcgttcaatgaacgcgtgcttcaggaagcggcggacaaatctaacccgctgattgaaaggatgcgtttcctggggatctattccaataaccttgatgagttctataaagtccgcttcgctgaactgaagcgacgcatcattattagcgaagaacaaggctccaactctcattcccgccatttactgggcaaaattcagtcccgggtgctgaaagccgatcaggaattcgacggcctctacaacgagctattgctggagatggcgcgcaaccagatcttcctgattaatgaacgccagctctccgtcaatcaacaaaactggctgcgtcattattttaagcagtatctgcgtcagcacattacgccgattttaatcaatcctgacactgacttagtgcagttcctgaaagatgattacacctatctggcggtggaaattatccgtggcgataccatccgttacgcgctgctggagatcccatcagataaagtgccgcgctttgtgaatttaccgccagaagcgccgcgtcgacgcaagccgatgattcttctggataacattctgcgttactgccttgatgatattttcaaaggcttctttgattatgacgcgctgaatgcctattcaatgaagatgacccgcgatgccgaatacgatttagtgcatgagatggaagccagcctgatggagttgatgtcttccagtctcaagcagcgtttaactgctgagccggtgcgttttgtttatcagcgcgatatgcccaatgcgctggttgaagtgttacgcgaaaaactgactatttcccgctacgactccatcgtccccggcggtcgttatcataattttaaagactttattaatttccccaatgtcggcaaagccaatctggtgaacaaaccactgccgcgtttacgccatatttggtttgataaagcccagttccgcaatggttttgatgccattcgcgaacgcgatgtgttgctctattatccttatcacacctttgagcatgtgctggaactgctgcgtcaggcttcgttcgacccgagcgtactggcgattaaaattaacatttaccgcgtggcgaaagattcacgcatcatcgactcgatgatccacgccgcacataacggtaagaaagtcaccgtggtggttgagttacaggcgcgtttcgacgaagaagccaacattcactgggcgaagcgcctgaccgaagcaggcgtgcacgttatcttctctgcgccggggctgaaaattcacgccaaactgttcctgatttcacgtaaagaaaacggtgaagtggtgcgttatgcacacatcgggaccgggaactttaacgaaaaaaccgcgcgtctttatactgactattcgttgctgaccgccgatgcgcgcatcaccaacgaagtacggcgggtatttaactttattgaaaacccataccgtccggtgacatttgattatttaatggtatcgccgcaaaactcccgccgcctattgtatgaaatggtggaccgcgagatcgccaacgcgcagcaagggctgcccagtggtatcaccctgaagctaaataaccttgtcgataaaggcctggttgatcgtctgtatgcggcctccagctccggcgtaccggttaatctgctggttcgcggaatgtgttcgctgatccccaatctggaaggcattagcgacaacattcgtgccatcagtattgttgaccgttaccttgaacatgaccgggtttatatttttgaaaatggcggcgataaaaaggtctacctttcttccgccgactggatgacgcgcaatattgattatcgtattgaagtggcgacgccgctgctcgatccgcgcctgaagcagcgggtactggacatcatcgacatattgttcagcgatacggtcaaagcacgttatatcgataaagaactcagtaatcgctacgttccccgcggcaatcgccgcaaagtacgggcgcagttggcgatttatgactacatcaaatcactcgaacaacctgaataa

SEQ ID NO: 11 - DNA, 30 bp, synthetic construct
  misc_feature 1..30: note = BpXylA forward primer
  source 1..30: mol_type = other DNA
atacatatgatggcaacaaaagagtacttt

SEQ ID NO: 12 - DNA, 35 bp, synthetic construct
  misc_feature 1..35: note = BpXylA reverse primer
  source 1..35: mol_type = other DNA
atagcggccgcgcaatacatatttacaatagcctc

SEQ ID NO: 13 - DNA, 38 bp, synthetic construct
  misc_feature 1..38: note = BpRpiB forward primer
  source 1..38: mol_type = other DNA
atgcatatgatgaaaacaataggacttgcatccgacca

SEQ ID NO: 14 - DNA, 35 bp, synthetic construct
  misc_feature 1..35: note = BpRpiB reverse primer
  source 1..35: mol_type = other DNA
atagcggccgctcgaacgggaattttgtcgatgcg

SEQ ID NO: 15 - DNA, 37 bp, synthetic construct
  misc_feature 1..37: note = AtDpe forward primer
  source 1..37: mol_type = other DNA
atacatatgaaacacggcatctattattcttactggg

SEQ ID NO: 16 - DNA, 29 bp, synthetic construct
  misc_feature 1..29: note = AtDpe reverse primer
  source 1..29: mol_type = other DNA
atagcggccgcgccaccaagaacgaagcg

SEQ ID NO: 17 - DNA, 29 bp, synthetic construct
  misc_feature 1..29: note = EcAraB forward primer
  source 1..29: mol_type = other DNA
atagagctcatggcgattgcaattggcct

SEQ ID NO: 18 - DNA, 29 bp, synthetic construct
  misc_feature 1..29: note = EcAraB reverse primer
  source 1..29: mol_type = other DNA
atagcggccgctagagtcgcaacggcctg

SEQ ID NO: 19 - DNA, 31 bp, synthetic construct
  misc_feature 1..31: note = EcPpk forward primer
  source 1..31: mol_type = other DNA
atacatatgggtcaggaaaagctatacatcg

SEQ ID NO: 20 - DNA, 33 bp, synthetic construct
  misc_feature 1..33: note = EcPpk reverse primer
  source 1..33: mol_type = other DNA
atagcggccgcttcaggttgttcgagtgatttg